(19) United States
(12) Patent Application Publication
(10) Pub. No.: US 2011/0275745 A1
(43) Pub. Date: Nov. 10, 2011

(54) MODIFIED MAGNESIUM OXYSULFATE FIBROUS PARTICLES AND POLYOLEFIN COMPOSITION CONTAINING THE SAME

(75) Inventors: Shinichi YAMAMOTO, Ube-shi (JP); Takashi KISHIMOTO, Ichihara-shi (JP)

(73) Assignee: UBE MATERIAL INDUSTRIES, LTD., Ube-shi (JP)

(21) Appl. No.: 13/186,029

(22) Filed: Jul. 19, 2011

Related U.S. Application Data
(63) Continuation of application No. 12/124,351, filed on May 21, 2008.

(51) Int. Cl. C08K 5/52; C07C 309/00; C08K 5/41; C07F 9/02

(52) U.S. Cl. 524/141; 562/23; 562/36; 524/156

(57) ABSTRACT

A modified magnesium oxysulfate fibrous particles in which the magnesium oxysulfate fibrous particles are treated on their surfaces with a nucleating agent such as a phosphoric acid compound, an organic acid compound or a sorbitol compound can be favorably employed as fillers for polyolefin composition.

### FIELD OF THE INVENTION

The present invention relates to magnesium oxysulfate fibrous particles which are favorably employable as fillers for polyolefin compositions.

### BACKGROUND OF THE INVENTION

Polyolefin resins are thermoplastic resin and employed for manufacturing various molded products such as molded products for automotive parts, electric and electronic parts, and mechanical parts.

Recently, there has been a demand for reducing weights of the automotive parts so as to improve fuel economy, without decreasing the necessary physical characteristics such as high rigidity (represented by high flexural modulas of elasticity) and high-impact properties. For the above-mentioned purpose, it has been studied to incorporate a polymer characteristic-modifying agent such as a filler (e.g., calcium carbonate, talc, mica or glass fiber) or a nucleating agent into polyolefin resins.

Japanese Patent Provisional Publication 62-91547 (JP 62-91547 A) describes a polypropylene resin composition comprising 100 weight parts of a polypropylene resin and 0.5 to 5 weight parts of a fibrous reinforcing agent having a mean width of 0.1-2 μm and a mean length of 20-150 μm. It is further described that 0.01-3 weight parts of a nucleating agent can be incorporated into the polypropylene resin composition. Examples of the fibrous reinforcing agents include fibrous magnesium oxide, fibrous magnesium oxysulfate, fibrous magnesium hydroxide, fibrous calcium titanate, glass fiber, fibrous calcium silicate, carbon fiber, rock wool, silicon nitride whisker, silicon carbide whisker, alumina silica glass fiber, and fibrous gypsum.

Japanese Patent Provisional Publication 6-220258 (JP 6-220258 A) describes a polyolefin composition comprising 100 weight parts of a polyolefin resin, 1 to 100 weight parts of an inorganic filler, and an organic acid selected from the group consisting of an aromatic phosphoric acid, a saturated aliphatic dicarboxylic acid, and an aromatic mono- or di-carboxylic acid. The organic acid is used in an amount of 0.1 to 10 weight % based on the amount of the inorganic filler. Examples of the inorganic fillers described include calcium titanate, barium titanate, magnesium sulfate, calcium sulfate, barium sulfate, magnesium sulfite, aluminum borate, zinc borate, calcium borate, barium borate, sodium borate, beryllium oxide, magnesium oxide, zinc oxide, magnesium hydroxide, and aluminum hydroxide. It is also described that the organic acid can be applied to the inorganic filler.

### SUMMARY OF THE INVENTION

The rigidity and impact resistance of polyolefin articles can be increased by the incorporation of a filler or a nucleating agent into the polyolefin resin. However, it is recently required that polyolefin articles employed as internal parts (such as instrumental panels) and external parts (such as bumpers) of automobiles have the following improved characteristics:

(1) increase of heat resistance represented by higher defection temperature under load; this is particularly important for the internal parts of automobiles because the internal parts are heated to 80° C. or higher (partially 90° C. or higher) in hot summer days,

(2) increase of dimensional stability represented by lower mean linear expansion coefficient of the resin articles; this is particularly important for the external parts of automobiles because the external parts are liable to largely expand its dimensions when they are placed under different temperature conditions, and

(3) decrease of weight without decrease of the rigidity and impact-resistance.

Accordingly, it is an object of the present invention to provide a filler which has a light weight and is favorably employed for improving the heat resistance (e.g., rigidity and impact-resistance at elevated temperatures) and dimensional stability of polyolefin resin molded articles.

As a result of studies, the present inventors have discovered that a fibrous magnesium oxysulfate (i.e., fibrous basic magnesium sulfate) having a relatively light weight and having been treated with a nucleating agent on its surface is favorably employable as a filler to be incorporated into polyolefin compositions to manufacture polyolefin molded product having the satisfactory characteristics, as compared with the case in which the fibrous magnesium oxysulfate and the nucleating agent are independently mixed with a polyolefin resin.

Accordingly, the present invention resides in a modified magnesium oxysulfate fibrous particles comprising magnesium oxysulfate fibrous particles treated on their surfaces with a nucleating agent.

The invention also resides in a polyolefin composition comprising 80 to 95 weight % of a polyolefin resin and 5 to 20 weight % of a modified magnesium oxysulfate fibrous particles comprising magnesium oxysulfate fibrous particles treated on their surfaces with a nucleating agent.

### PREFERRED EMBODIMENTS OF THE INVENTION

In the modified magnesium oxysulfate fibrous particles of the invention, the nucleating agent is preferably attached to the magnesium oxysulfate fibrous particles in an amount of 0.1 to 5 weight parts, more preferably 0.2 to 3 weight parts, per 100 weight parts of the fibrous particles.

In the modified magnesium oxysulfate fibrous particles of the invention, the nucleating agent can be a nucleating agent which is conventionally known as a nucleating agent for the use in the manufacture of polyolefin resin molded articles. The nucleating agent preferably is a phosphoric acid compound, an organic acid compound or a sorbitol compound. The phosphoric acid preferably is a salt of an aromatic phosphoric acid. The organic acid compound can be an organic acid, a salt of the organic acid, an ester of the organic acid and an anhydrides of the organic acid. The organic acid is preferably selected from the group consisting of aliphatic monocarboxylic acids, aliphatic dicarboxylic acids, aromatic monocarboxylic acids, aromatic dicarboxylic acids, and organic phosphoric acids.

The modified magnesium oxysulfate fibrous particles of the invention comprises magnesium oxysulfate fibrous particles which are so treated on their surfaces with a nucleating agent that the nucleating agent is preferably attached onto the magnesium oxysulfate fibrous particles. The nucleating agent is preferably attached to the magnesium oxysulfate fibrous particles in an amount of 0.1 to 5 weight parts, preferably 0.2 to 3 weight parts, per 100 weight parts of the fibrous particles.

The magnesium oxysulfate fibrous particles to be treated on their surfaces with a nucleating agent in the invention are known fibrous (or acicular) particles of magnesium oxysulfate (or basic magnesium sulfate) represented by MgSO₄.5Mg(OH)₃.3H₂O. The magnesium oxysulfate fibrous particles have a true specific gravity of approx. 2.3 which is relatively light, as compared with other fibrous particles such as calcium titanate fibrous particles and calcium carbonate fibrous particles.

The magnesium oxysulfate fibrous particles to be modified in the invention preferably have a BET specific surface area of 7 to 15 m²/g, a mean length of 8 to 30 μm, a mean width (or thickness) of 0.5 to 1.0 μm, and a mean aspect ratio (mean length/mean width) of 10 to 50. Such magnesium oxysulfate fibrous particles can be prepared by hydrothermal reaction of magnesium hydroxide and magnesium sulfate.

The nucleating agents to be employed in the invention can be phosphoric acids, salts thereof, organic acids, salts thereof and sorbitols. The phosphoric acids and salts thereof are most preferred.

The organic acid can be an aliphatic monocarboxylic acid, an aliphatic dicarboxylic acid, an alicyclic monocarboxylic acid, an alicyclic dicarboxylic acid, an aromatic monocarboxylic acid, an aromatic dicarboxylic acid, an organic phosphoric acid, phenylacetic acid, diphenylacetic acid, isonicotinic acid or benzenesulfonic acid. The nucleating agent can be a salt of an organic acid or an organic acid ester. The salt can be a sodium salt, a potassium salt, an aluminum salt, a magnesium salt or an amine salt. The ester can be a lower alkyl ester such as methylester or ethylester.

The aliphatic dicarboxylic acid preferably is a saturated aliphatic dicarboxylic acid having 3 to 12 carbon atoms. Examples of the saturated aliphatic di-carboxylic acids include malonic acid, succinic acid, glutaric acid, adipic acid, azelaic acid and sebacic acid.

Examples of the aromatic mono- or dicarboxylic acids include benzoic acids, naphthoic acids, and cinnamic acids. In more detail, benzoic acid, methylbenzoic acid, ethylbenzoic acid, isopropylbenzoic acid, p-tert-butylbenzoic acid, phenylbenzoic acid, α-naphthoic acid, β-naphthoic acid, cinnamic acid, methylcinnamic acid, ethylcinnamic acid, isopropylcinnamic acid, tert-butylcinnamic acid, phthalic acid, isophthalic acid, and terephthalic acid can be mentioned.

Examples of the aliphatic carboxylic acids include cyclohexanecarboxylic acid, cyclopentanecarboxylic acid, hexanoic acid, isohexanoic acid and abietic acid.

Examples of the aromatic phosphoric acids can be phenylphosphoric acids which can be substituted 1 to 3 alkyl groups having 1 to 4 carbon atoms and 2,2′-methylene, ethylidene, propylidene or butylidene derivatives of the phenylphosphoric acids. In more detail, phenylphosphoric acid, t-butylphenylphosphoric acid, diphenylphosphoric acid, bis(t-butylphenyl)phosphoric acid, bis(d-t-butylphenyl)phosphoric acid, 2,2′-methylenebis(dimethylphenyl)phosphoric acid, 2,2′-methylenebis(di-t-butylphenyl)phosphoric acid, sodium 2,2′-methylene bis(4,6-di-tert-butylphenyl)phosphate and sodium bis(4-tert-butylphenyl)phosphate can be mentioned as the aromatic phosphoric acids and salts thereof.

Examples of the sorbitol compounds include bis(p-methylbenzylidene)sorbitol, bis(alkylbenzylidene)sorbitol, bis(p-ethylbenzylidene)sorbitol, 1,3,2,4-dibenzylidenesorbitol, 1,3,2,4-di(methoxybenzylidene)sorbitol and 1,3,2-4-di(ethoxydibenzylidene)sorbitol.

The nucleating agent preferably is an aromatic phosphoric acid or its salt which can easily reacts with the hydroxide group of the magnesium oxysulfate fibrous particles, whereby the nucleating agent is firmly attached to the surface of the magnesium oxysulfate fibrous particles.

The nucleating agents can be employed singly or in combination.

The magnesium oxysulfate fibrous particles can be treated with the nucleating agent by adding the nucleating agent or its anhydride (e.g., benzoic anhydride) to an aqueous slurry of the magnesium oxysulfate fibrous particles, stirring the slurry, dehydrating the slurry using a known dehydrating apparatus such as a centrifugal separator or a vacuum filter to give a cake of the modified fibrous particles, and drying the cake. The nucleating agent is preferably employed in an amount of 0.1 to 5 weight parts, more preferably in an amount of 0.2 to 3 weight parts, based on the amount of the magnesium oxysulfate fibrous particles.

The modified magnesium oxysulfate fibrous particles of the invention, that is, the magnesium oxysulfate fibrous particles treated on their surface with the nucleating agent, can be favorably mixed with a polyolefin resin, to give a molded polyolefin resin product having the improved characteristics such as high heat-resistance, high dimensional stability, rigidity and high impact resistance.

The polyolefin resin can be a homopolymer or a copolymer of α-olefin such as ethylene, propylene, butene, pentene, hexene, 4-methyl-1-pentene, or octane. The α-olefin can be copolymerized or graft-copolymerized with other unsaturated monomers such as a vinylester (e.g., vinyl acetate or vinyl butyrate), an unsaturated carboxylic acid or its derivative (e.g., acrylic acid, maleic anhydride, methyl methacrylate, or ethyl acrylate), and an unsaturated aromatic monomer (e.g., styrene or α-methylstyrene). The copolymers can be saponified or converted into their metal salts. In more detail, ultra-low density polyethylene, low density polyethylene, linear low density polyethylene, medium density polyethylene, high density polyethylene, polypropylene, polybutene, poly-3-methylpentene-1, copolymers of propylene with other α-olefins, random, block or graft copolymers of ethylene with vinylesters, unsaturated carboxylic acids, their derivatives, or unsaturated aromatic monomers. The copolymers can be further modified.

The modified magnesium oxysulfate fibrous particles of the invention can be mixed with polyolefin resin in an amount of 5 to 20 weight %, preferably 5 to 15 weight %, in the mixed polyolefin composition, in such manner that the modified magnesium oxysulfate fibrous particles are uniformly dispersed in the mixed polyolefin composition. For the purpose of dispersing the modified magnesium oxysulfate fibrous particles uniformly in the mixed polyolefin composition, the modified magnesium oxysulfate fibrous particles and a polyolefin resin are simultaneously or sequentially placed in a kneader and kneaded under heating. Otherwise, 50 to 90 weight parts, preferably 60 to 80 weight parts, of the modified magnesium oxysulfate fibrous particles and 10 to 50 weight parts, preferably 20 to 40 weight parts, of the polyolefin resin are first mixed and kneaded under heating to give a master batch having a high concentration of the modified magnesium oxysulfate fibrous particles, and subsequently the master batch is mixed with an additional amount of the polyolefin resin and kneaded to give the desired polyolefin composition containing the predetermined amount of the modified magnesium oxysulfate fibrous particles. The kneader can be one of known kneaders such as an extruder (e.g., a monoaxial extruder or a biaxial extruder), a biaxial continuous mixer, a Banbury mixer, a super-mixer, a mixing roller, a kneader, or a Brabender Plastograph.

The polyolefin composition of the invention which contains the modified magnesium oxysulfate fibrous particles may further contain other additives such as heat stabilizers, photo stabilizers, plasticizers, cross-linking agents, oxidation inhibitors, fire retardant, pigments, dyes, lubricants, antistatic agents, releasing agents, perfumes, inorganic fillers such as calcium carbonate, talc, mica and glass fiber, elastic material, thermoplastic elastomers, or thermoplastic resin modified by elastomer.

The polyolefin composition of the invention which contains the modified magnesium oxysulfate fibrous particles can be molded by a known molding method using an injection molding machine to give molded resin articles. The molded resin articles can be installed in automobiles, electric-electronic devices and various apparatuses, particularly can be favorably employed in automobiles as internal parts (e.g., instrument panels) and external parts (e.g., bumper).

The present invention is further described by the following examples.

In the following examples, the deflection temperature under load, mean linear expansion coefficient (in machine direction—MD), flexural modulus of elasticity, Izod impact strength and density were measured by the below-described methods.

(1) The deflection temperature under load was measured by the method defined in JIS-K-7207 (1995). The specimen had sizes of 127.0 mm×12.5 mm×3.0 mm. The higher deflection temperature means a higher heat resistance.

(2) The mean linear expansion coefficient was measured on a long specimen along its length direction by the method defined in JIS-K-7197 (1991). The specimen had sizes of 15.0 mm×6.25 mm×3.0 mm. The higher mean linear expansion coefficient means a better dimensional stability.

(3) The flexural modulus of elasticity was measured by the method defined in JIS-K-7171 (1994). The higher flexural modulus of elasticity means a higher rigidity.

(4) The Izod impact strength was measured by the method defined in JIS-K-7110 (1984). The specimen had sizes of 63.5 mm×12.5 mm×3.0 mm and a notch at the center along the length direction.

(5) The density was measured by a known method.

### Example 1

In 100 L of water was dispersed 2,000 g of magnesium oxysulfate fibrous particles (MOS-HIDE, available from Ube Material Industries Co., Ltd., BET specific surface area: 9 m²/g, mean fiber length: 15 μm, mean fiber width: 0.5 μm, mean aspect ratio: 30), to give a fibrous magnesium oxysulfate slurry. To this slurry was added 20 g of sodium 2,2′-methylene bis(4,6-di-tert-butylphenyl)phosphate (ADEKA STUB NA-11, available from Asahi Denka Co., Ltd.), and the slurry was stirred for one hour. The slurry was then dehydrated on a vacuum filter at a linear pressure of 5 kg/cm² to give a cake containing 50 wt. % of water. The cake was dried in a box dryer to obtain the modified magnesium oxysulfate fibrous particles.

In a kneader (PCM-30, available from Ikegai Corporation), 89.5 weight parts of ethylene-propylene block copolymer (ethylene content: 8 wt. %, MFI: 30 g/10 min.), 10 weight parts of the modified magnesium oxysulfate fibrous particles, 0.3 weight part of lubricant (calcium stearate), and 0.2 weight part of oxidation inhibitors (0.1 weight part of Irgafos available from Ciba-Geigy and 0.1 weight part of Irganos available from Ciba-Geigy) were melted and kneaded to obtain a polyolefin composition.

The polyolefin composition was injected from an injection molding machine to give a molded article for measurements of the physical characteristics. The measured physical characteristics are set forth in Table 1.

### Comparison Example 1

A polyolefin composition was prepared in the same manner as in Example 1 except that the modified magnesium oxysulfate fibrous particles was replaced with the same amount of the unmodified magnesium oxysulfate fibrous particles and 0.1 weight part of sodium 2,2′-methylene bis(4,6-di-tert-butylphenyl)phosphate was separately added.

The polyolefin composition was injected from an injection molding machine to give a molded article for measurements of the physical characteristics. The measured physical characteristics are set forth in Table 1.

### Example 2

The procedures of Example 1 were repeated except that sodium 2,2′-methylene bis(4,6-di-tert-butylphenyl)phosphate was replaced with the same amount of p-tert-butylbenzoic acid, to obtain the modified magnesium oxysulfate fibrous particles.

The modified magnesium oxysulfate fibrous particles were mixed with 89.5 weight parts of the ethylene-propylene block copolymer, 0.3 weight part of the lubricant, and 0.2 weight part of oxidation inhibitors and melted and kneaded in the same manner as in Example 1 to obtain a polyolefin composition.

The polyolefin composition was injected from an injection molding machine to give a molded article for measurements of the physical characteristics. The measured physical characteristics are set forth in Table 1.

### Comparison Example 2

A polyolefin composition was prepared in the same manner as in Example 1 except that the modified magnesium oxysulfate fibrous particles was replaced with the same amount of the unmodified magnesium oxysulfate fibrous particles and 0.1 weight part of p-tert-butylbenzoic acid was separately added.

The polyolefin composition was injected from an injection molding machine to give a molded article for measurements of the physical characteristics. The measured physical characteristics are set forth in Table 1.

### Example 3

The procedures of Example 1 were repeated except that sodium 2,2′-methylene bis(4,6-di-tert-butylphenyl)phosphate was replaced with the same amount of benzoic acid, to obtain the modified magnesium oxysulfate fibrous particles.

The modified magnesium oxysulfate fibrous particles were mixed with 89.5 weight parts of the ethylene-propylene block copolymer, 0.3 weight part of the lubricant, and 0.2 weight part of oxidation inhibitors and melted and kneaded in the same manner as in Example 1 to obtain a polyolefin composition.

The polyolefin composition was injected from an injection molding machine to give a molded article for measurements of the physical characteristics. The measured physical characteristics are set forth in Table 1.

### Comparison Example 3

A polyolefin composition was prepared in the same manner as in Example 3 except that the modified magnesium oxysulfate fibrous particles was replaced with the same amount of the unmodified magnesium oxysulfate fibrous particles and 0.1 weight part of benzoic acid was separately added.

The polyolefin composition was injected from an injection molding machine to give a molded article for measurements of the physical characteristics. The measured physical characteristics are set forth in the following Table 1.

TABLE 1

| Example | (1) | (2) | (3) | (4) | (5) |
|---|---|---|---|---|---|
| Ex. 1 | 120 | 0.74 × 10⁻⁴ | 2320 | 5.8 | 0.960 |
| Com. 1 | 115 | 0.84 × 10⁻⁴ | 1790 | 5.4 | 0.961 |
| Ex. 2 | 118 | 0.71 × 10⁻⁴ | 2110 | 5.9 | 0.960 |
| Com. 2 | 112 | 0.86 × 10⁻⁴ | 1940 | 5.2 | 0.959 |
| Ex. 3 | 116 | 0.75 × 10⁻⁴ | 2100 | 5.8 | 0.961 |
| Com. 3 | 112 | 0.85 × 10⁻⁴ | 1940 | 5.4 | 0.960 |

Remarks:
(1) Deflection temperature under load (° C.)
(2) Mean linear expansion coefficient (/° C.)
(3) Flexural modulus of elasticity (MPa)
(4) Izod impact strength (kJ/m²)
(5) Density (g/cm³)

It is understood from the data in Table 1 that the polyolefin articles of Examples 1 to 3 which were manufactured using the modified magnesium oxysulfate fibrous particles of the invention were improved in their heat resistance, dimensional stability, rigidity and impact strength, as compared with the polyolefin articles of Comparison Examples 1 to 3 which were manufactured using the unmodified magnesium oxysulfate fibrous particles and the separately added nucleating agent, at the essentially same density.

What is claimed is:

1. Modified magnesium oxysulfate fibrous particles comprising magnesium oxysulfate fibrous particles treated on their surfaces with a nucleating agent which is selected from the group consisting of phosphoric acid compounds, aromatic monocarboxylic acid, aromatic dicarboxylic acid and sorbitol compounds.

2. The modified magnesium oxysulfate fibrous particles of claim 1, in which the nucleating agent is attached to the magnesium oxysulfate fibrous particles in an amount of 0.1 to 5 weight parts per 100 weight parts of the fibrous particles.

3. The modified magnesium oxysulfate fibrous particles of claim 2, in which the nucleating agent is attached to the magnesium oxysulfate fibrous particles in an amount of 0.2 to 3 weight parts per 100 weight parts of the fibrous particles.

4. The modified magnesium oxysulfate fibrous particles of claim 1, in which the nucleating agent is a phosphoric acid compound.

5. The modified magnesium oxysulfate fibrous particles of claim 4, in which the phosphoric acid compound is a salt of an aromatic phosphoric acid.

6. The modified magnesium oxysulfate fibrous particles of claim 5, in which the salt of aromatic phosphoric acid is selected from the group consisting of sodium 2,2′-methylene bis(4,6-di-tert-butylphenyl)phosphate and sodium bis(4-tert-butylphenyl)phosphate.

7. A polyolefin composition comprising 80 to 95 weight % of a polyolefin resin and 5 to 20 weight % of modified magnesium oxysulfate fibrous particles comprising magnesium oxysulfate fibrous particles treated on their surfaces with a nucleating agent which is selected from the group consisting of phosphoric acid compounds, aromatic monocarboxylic acid, aromatic dicarboxylic acid and sorbitol.

8. The polyolefin composition of claim 7, in which the nucleating agent is attached to the magnesium oxysulfate fibrous particles in an amount of 0.1 to 5 weight parts per 100 weight parts of the fibrous particles.

9. The polyolefin composition of claim 8, in which the nucleating agent is attached to the magnesium oxysulfate fibrous particles in an amount of 0.2 to 3 weight parts per 100 weight parts of the fibrous particles.

10. The polyolefin composition of claim 7, in which the nucleating agent is a phosphoric acid compound.

11. The polyolefin composition of claim 10, in which the phosphoric acid compound is a salt of an aromatic phosphoric acid.

12. The polyolefin composition of claim 11, in which the salt of aromatic phosphoric acid is selected from the group consisting of sodium 2,2′-methylene bis(4,6-di-tert-butylphenyl)phosphate and sodium bis(4-tert-butylphenyl)phosphate.

13. The modified magnesium oxysulfate fibrous particles of claim 1, in which the nucleating agent is an aromatic dicarboxylic acid.

14. The polyolefin composition of claim 7, in which the nucleating agent is an aromatic dicarboxylic acid.
